(19) United States
(12) Patent Application Publication
(10) Pub. No.: US 2016/0160229 A1
(43) Pub. Date: Jun. 9, 2016

(54) METHODS OF PRODUCING ANTIBODY-RICH CANNABIS AND HONEYSUCKLE PLANTS

(71) Applicant: Sadia "Ross" Barrameda, Discovery Bay, CA (US)

(72) Inventor: Sadia "Ross" Barrameda, Discovery Bay, CA (US)

(73) Assignees: THC Farmaceuticals, Inc., Scottsdale, AZ (US); Sadia "Ross" Barrameda, Discovery Bay, CA (US)

(21) Appl. No.: 14/564,672

(22) Filed: Dec. 9, 2014

(51) Int. Cl. C12N 15/82; A61K 36/355; A61K 39/395; A61K 36/185

(52) U.S. Cl. CPC C12N 15/8258; A61K 36/185; A61K 36/355; A61K 39/395; A61K 2039/505

(57) ABSTRACT

A method of producing antibody-rich cannabis and honeysuckle plants, and receiving the antibodies therefrom within a human body, is described. Antibody proteins are transfected or transduced to the DNA of one or more cells of a cannabis or honeysuckle seed. As the cannabis or honeysuckle seed grows into a plant, the plant cells reproduce the antibody protein within the DNA into a plurality of cells. A human consumes the cannabis or honeysuckle plant. The digestive system of the human separates the antibody proteins from the DNA. Finally, the circulatory system of the human distributes the antibody proteins throughout the body.

### FIELD OF THE INVENTION

This invention relates generally to the production of antibody-rich cannabis and honeysuckle plants.

### BACKGROUND OF THE INVENTION

Antibodies are the manifestation of the body's response to potential harmful pathogens, such as bacteria and viruses. Antibodies, or immunoglobulin, are Y-shaped glycoproteins produced by the immune system. Each tip of the “Y” contains a paratope that is adapted to bind with a particular epitope of a pathogen. The antibody may target the pathogen for attack by other components of the immune system or may neutralize the pathogen directly. Even though effective antibodies are created by human immune systems, it is impractical to receive antibodies from a human for any form of mass-production.

Antibodies are also produced through cultures of hybridoma cells, for example, that may be grown to contain the antibodies. Once the cells are grown, the antibodies may be removed by standard methods, such as ammonium sulphate precipitate. The process of growing the antibodies in cultures and isolating them is impractical for large-scale production of antibodies.

Antibodies may be grown in plant leaves as well, as is shown in U.S. Pat. No. 8,624,080 (Werner et al.) Werner discloses a process of producing a protein, by introducing to a plant a nucleotide sequence and an inducible promoter linked to the nucleotide sequence, the nucleotide sequence encoding a polymerase and the protein. Next, the promoter is induced and produces the protein in the plant cells. Using a plant to grow the antibodies reduces the risk of contamination by mammalian viruses, however the process of growing the antibodies is a long one—first the plant is grown to a leaf-bearing stage, and then each plant must be treated with an engineered plant virus that has been tricked in to producing significant amounts of antibody proteins. Once the antibodies have been grown in the leaves, the proteins must be extracted from the plant cells and isolated in order to be converted to pharmaceutical use for human consumption. The process takes several months, and even then, only small amounts of the antibodies are produced.

As an example, the influenza virus antibodies may be grown in plants after being treated with a virus as above. One of the problems in regard to antibodies for the influenza virus is the many variations of the virus, wherein antibodies for one strain are not effective for another, and wherein multiple strains are spreading at any given time. When determining which “deactivated” virus to inject for a “flu shot”, scientists merely guess based on the frequency and location of influenza cases, and are frequently wrong.

As a further example, the Ebola virus has been combatted recently using a drug called ZMapp, which contains three of the most effective antibodies against Ebola so far discovered. The chimeric monoclonal antibodies (mAbs) are “MB-003”, ZMab, c2G4 and c4G7. ZMapp is manufactured in the leaves of a certain tobacco strain particularly susceptible to plant viruses. For the antibodies to be produced in the plant genes coding for the chimeric mAbs are inserted into viral vectors, and tobacco leaves are infected with the viral vector encoding for the antibodies, using Agrobacterium cultures. The tobacco plant is grown for a period of weeks, after which point the antibodies are extracted from the leaves and isolated for manufacture.

In any of the prior art antibody production methods above, the need to extract and isolate the antibodies is a costly and time-consuming part of the process. In addition, the process of growing the unadulterated plants to a certain maturity and infecting the mature plant with a virus to introduce the protein to the plant

Furthermore, cannabis is beneficial in medicine as reducing stress and pain, as well as nausea in chemotherapy patients, for example. Cannabis can be easily consumed by vaporizing, smoking, eating or otherwise ingesting it. The two most medically active strains of Cannabis are Cannabis Sativa and Cannabis Indica. In addition, cannabis contains a number of beneficial antioxidants and proteins.

Honeysuckle contains naturally-occurring antiviral agents such as MIR2911, which represses Influenza “A” viruses (IAV) by targeting two specific genes that have been identified as essential for influenza replication, PB2 and NS1. Honeysuckle may be easily consumed in tea, or by eating or inhaling the vapors or fumes thereof.

Therefore, there is a need for a less expensive, more cost-effective means of achieving the benefits of antibodies without the need for extraction and isolation, and further with the medical benefits of cannabis or honeysuckle in their ease of consumption and beneficial medical effects such as pain, nausea reduction and antiviral properties.

### SUMMARY OF THE INVENTION

The foregoing, and other features and advantages of the invention, will be apparent from the following, more particular description of the preferred embodiments of the invention, the accompanying drawings, and the claims.

A method of producing antibody-rich cannabis plants and receiving the antibodies therefrom within a human body is described, comprising the steps of transfecting antibody proteins to the DNA of one or more cells of a cannabis seed; as the cannabis seed grows into a cannabis plant, the plant cells reproducing the antibody protein within the DNA into a plurality of cells; a human body consuming the cannabis plant; a digestive system of the human body separating the antibody proteins from the DNA; and a circulatory system of the human body distributing the antibody proteins throughout the body.

In an embodiment of the invention, the transfecting of antibody proteins is a chemical transfecting using a chemical selected from the group consisting of cyclodextrin, polymers, liposomes or nanoparticles.

In a further embodiment, the transfecting of antibody proteins is a non-chemical transfecting selected from the group consisting of electroporation, sonoportation, cell-squeezing, optical transfection and protoplast fusion. In an embodiment, consuming the plant comprises eating, drinking, smoking or inhaling vapors of the plant.

Further, a method of producing antibody-rich cannabis plants and receiving the antibodies therefrom within a human body is described, comprising the steps of virally-transducing antibody proteins to the DNA of one or more cells of a cannabis plant so the antibody proteins are stably inherited by the cells; a human body consuming the cannabis plant; a digestive system of the human body separating the antibody proteins from the DNA; and a circulatory system of the human body distributing the antibody proteins throughout the body.

In an embodiment the additional step of inducing a lysogen is performed, which results in the antibody protein being excised from the DNA. Furthermore, the additional step of the cell releasing phage particles may be performed. In an embodiment consuming the plant comprises eating, drinking, smoking or inhaling vapors of the plant.

Further, a method of producing antibody-rich honeysuckle plants and receiving the antibodies therefrom within a human body is described, comprising the steps of transfecting or transducing antibody proteins to the DNA of one or more cells of a honeysuckle plant; as the honeysuckle plant grows, the plant cells reproducing the antibody protein within the DNA into a plurality of cells; a human body consuming the plant; a digestive system of the human body separating the antibody proteins from the DNA; and a circulatory system of the human body distributing the antibody proteins throughout the body. In an embodiment, the honeysuckle contains one or more antiviral compounds, wherein the antiviral compounds benefit the effect of the antibody proteins within the body.

### BRIEF DESCRIPTION OF THE FIGURES

For a more complete understanding of the present invention, the objects and advantages thereof, reference is now made to the ensuing descriptions taken in connection with the accompanying drawings briefly described as follows.

FIG. 1 is a flowchart showing steps of the method of producing antibody rich cannabis or honeysuckle plants from a seed; and

FIG. 2 is a flowchart showing steps of a method of producing antibody-rich plant parts.

### DETAILED DESCRIPTION OF PREFERRED EMBODIMENTS

In one embodiment of the present invention, antibody proteins are transfected to the leaves of the cannabis or honeysuckle plant. The transfection inserts the protein of the antibody into the seeds or leaves, by chemical-based transfection, non-chemical methods, particle-based methods or viral methods.

Chemical-based transfection may involve the use of cyclodextrin, polymers, liposomes or nanoparticles. One example is the use of calcium phosphate, combined with calcium chloride solution containing the protein to be transfected. When the solutions are combined, a precipitate forms having the antibody protein on its surface. When the precipitate is introduced to cells, the cells take up the precipitate and protein. Alternatively, dendrimers may be used to bind the protein and bring it into the cell. Liposomes may also carry the protein, and may fuse to a cell membrane and introduce the protein into the cell.

In non-chemical methods, electroporation may be used wherein an increase in the permeability of a cell membrane may be brought about with a short pulse of an electric field. Cell-squeezing enables delivery of molecules by gently squeezing the membrane of the cell. Sonoporation uses ultrasound to induce pore formation in cell membranes. Optical transfection involves burning a small hole within the plasma membrane for the introduction of the antibody proteins. Protoplast fusion is a technique in which bacterial cells are treated with lysosome to remove the cell wall, and then fusogenic agents are used to fuse the protoplast carrying the protein of interest with the target cell.

Transfecting the antibody protein into the cells of the seeds of a cannabis or honeysuckle plant enables the plant to grow, all the while containing the protein of the antibody. As the cells reproduce and the plant grows, each of the new cells contains the antibody protein. The plant eventually may produce its own seeds, which can be replanted and grow on their own, again containing the antibody protein. As an alternative the leaves of a mature plant may be treated in order to modify the DNA of the cells to produce antibodies.

In viral transduction, adenoviral vectors can be useful for viral transfection as they are able to transfer genes to a wide variety of cells. Viral transduction may be used to stably introduce the antibody protein to the plant genome, so that the antibody is stably inherited by the plant's DNA and reproduced as the plant cells reproduce. Once the transduction is complete, the antibody is a component of most or all of the plant cells. Depending on the genetic expression of the transduced genes, the plant may be fertile or infertile after transduction.

Viral transduction happens through either the lytic cycle or lysogenic cycle. If the lysogenic cycle is adopted, the phage chromosome containing the antibody protein is integrated (by covalent bonds) into the cell chromosome, where it can remain dormant for thousands of generations. The cell continues to live and reproduce normally, with the new DNA containing the phage chromosome. If the lysogen is induced (by UV light or chemicals for example), the phage genome is excised from the bacterial chromosome and initiates the lytic cycle, which culminates in lysis of the cell and the release of phage particles, to infect other cells. The lytic cycle leads to the production of new phage particles that are released by lysis of the host.

The virus harnesses the cell's reproductive machinery to reproduce multiple copies of itself and the antibody protein within the cell. The antibody and virus are then released from the cell as the cell dies, with the result that the plant is filled with antibody proteins.

Once produced within a plant, the antibodies would not require extraction and isolation, both costly and time consuming parts of the process. The cannabis can be easily consumed in its natural state and therefore presents an optimal antibody delivery system. Consumption of the antibody-containing cannabis would be a straightforward matter of inhaling or ingesting. Honeysuckle would preferably be consumed in a tea. Of the antibodies produced within the plant cell, some antibodies may exist on their own, already isolated within the plant material. On ingestion by a human, the antibodies that are not broken down through the digestive process pass into the bloodstream. Other antibodies exist as part of the cell DNA within the plant. It has been shown that, while the human digestive system breaks down some of the DNA and RNA that is digested, some of it remains intact and is absorbed through the digestive system and circulates within the blood stream, intact.

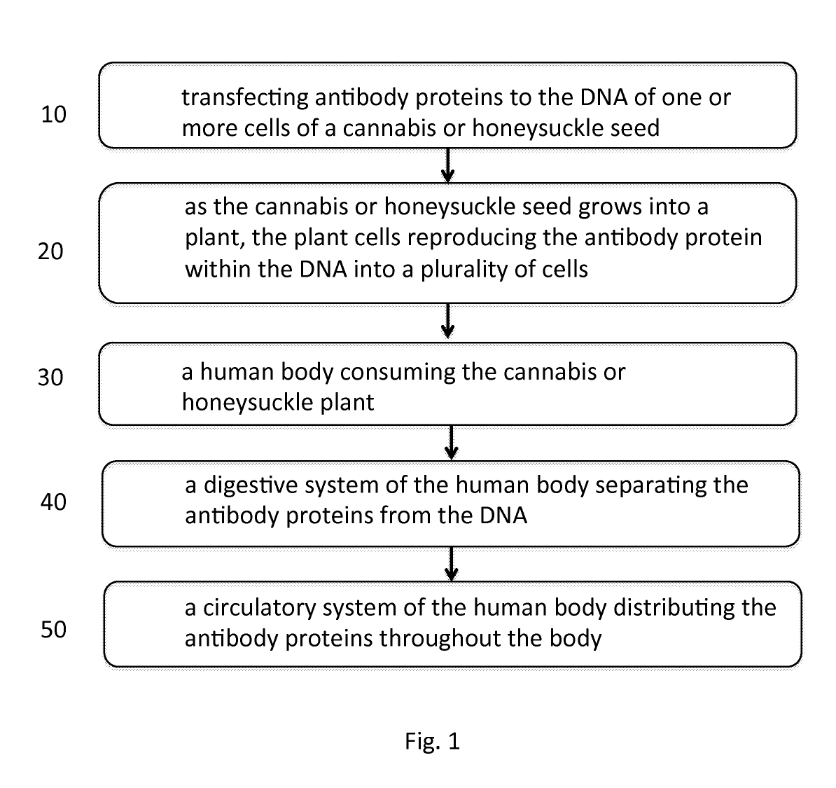

With reference to FIG. 1, a method of producing antibody-rich cannabis or honeysuckle plants and receiving the antibodies therefrom within a human body is described. In step 10, antibody proteins are transfected or transduced to the DNA of one or more cells of a cannabis or honeysuckle seed. In step 20, as the seed grows into a plant, the plant cells reproduce the antibody protein within the DNA into a plurality of cells. In step 30, a human consumes the plant, by eating, drinking or inhaling the plant parts. In step 40 the digestive system of the human separates the antibody proteins from the DNA. In step 50, the circulatory system of the human distributes the antibody proteins throughout the body.

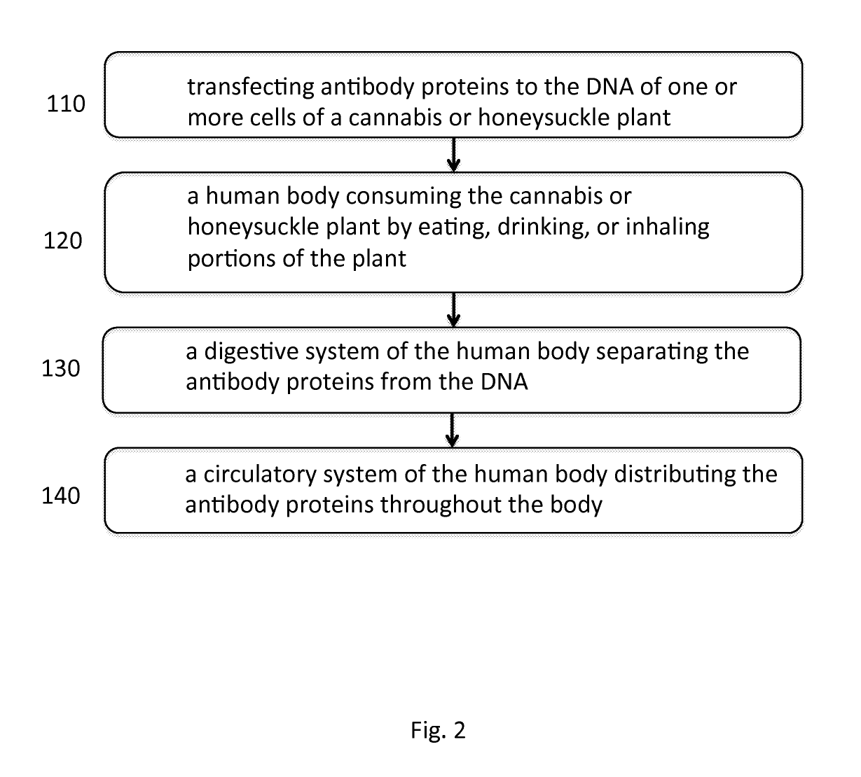

With reference to FIG. 2, a method of producing antibody-rich cannabis or honeysuckle plants by transduction and receiving the antibodies therefrom within a human body is described. In step 110, antibody proteins are transduced to the DNA of one or more cells of a cannabis or honeysuckle plant, and the plant cells reproduce normally to replicate the antibody proteins. In step 120, a human consumes the plant, by eating, drinking or inhaling the plant parts. In step 130 the digestive system of the human separates the antibody proteins from the DNA. In step 140, the circulatory system of the human distributes the antibody proteins throughout the body.

Depending on the antibody protein, it may break down as a result of the potential heat involved in methods of ingestion such as smoking or vaporizing. However, if the heat is disruptive to the protein's structure, then lower-heat or non-heat methods of consumption such as vaporizing or eating the cannabis may be used. The antibodies contained within the cannabis are separated naturally by the digestive system, and allow the antibodies to enter the bloodstream and combat the virus.

Smoking or vaporizing and inhaling the cannabis or honeysuckle causes the components to travel directly to the brain. Ingesting cannabis causes THC to be metabolized by the liver and converted to 11-hydroxy-THC which is particularly effective at crossing the blood-brain barrier. The pain-blocking effects and stress-blocking effects of THC last longer when eaten rather than smoked or vaporized. Inhaling honeysuckle causes the antiviral components to be introduced into the body, as they are not affected by the heat of vaporizing or smoking.

The additional benefits of consuming the antibodies within cannabis would include a reduction in stress, pain and nausea, as well as antioxidant properties of the cannabis plant. The impact of viruses on the human immune system is known to cause nausea, and cannabis addresses this nausea. Honeysuckle's natural antiviral properties found in MIR2911 and other compounds would benefit the human consuming it by bolstering the effect of the antibodies that are reproduced within the plant through this method.

In addition, cannabis and hemp contain edestin and albumin proteins. Edestin is found only in hemp seeds, and is similar to the body's own globular proteins found in blood plasma. Edestin is active in DNA repair and produces antibodies for a healthy immune system. Albumin is another high quality protein similar to that found in egg whites, and is also used for DNA repair. The availability of the edestin and albumin enables DNA repair, to correct any defects or mutations produced in the transfection transduction processes, resulting in the cannabis plant being a more stable platform for transfection or transduction.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A method of producing antibody-rich cannabis plants and receiving the antibodies therefrom within a human body, comprising the steps of:
a. transfecting antibody proteins to the DNA of one or more cells of a cannabis seed;
b. as the cannabis seed grows into a cannabis plant, the plant cells reproducing the antibody protein within the DNA into a plurality of cells;
c. a human body consuming the cannabis plant;
d. a digestive system of the human body separating the antibody proteins from the DNA; and
e. a circulatory system of the human body distributing the antibody proteins throughout the body.

2. The method of claim 1, wherein the transfecting of antibody proteins is a chemical transfecting using a chemical selected from the group consisting of cyclodextrin, polymers, liposomes or nanoparticles.

3. The method of claim 1, wherein the transfecting of antibody proteins is a non-chemical transfecting selected from the group consisting of electroporation, sonoportation, cell-squeezing, optical transfection and protoplast fusion.

4. The method of claim 1 wherein consuming the plant comprises eating, drinking, smoking or inhaling vapors of the plant.

5. A method of producing antibody-rich cannabis plants and receiving the antibodies therefrom within a human body, comprising the steps of:
a. virally-transducing antibody proteins to the DNA of one or more cells of a cannabis plant so the antibody proteins are stably inherited by the cells;
b. a human body consuming the cannabis plant;
c. a digestive system of the human body separating the antibody proteins from the DNA; and
d. a circulatory system of the human body distributing the antibody proteins throughout the body.

6. The method of claim 5, further comprising the step of inducing a lysogen, wherein the antibody protein is excised from the DNA.

7. The method of claim 6 further comprising the step of the cell releasing phage particles.

8. The method of claim 5 wherein consuming the plant comprises eating, drinking, smoking or inhaling vapors of the plant.

9. A method of producing antibody-rich honeysuckle plants and receiving the antibodies therefrom within a human body, comprising the steps of:
a. transfecting or transducing antibody proteins to the DNA of one or more cells of a honeysuckle plant;
b. as the honeysuckle plant grows, the plant cells reproducing the antibody protein within the DNA into a plurality of cells;
c. a human body consuming the plant;
d. a digestive system of the human body separating the antibody proteins from the DNA; and
e. a circulatory system of the human body distributing the antibody proteins throughout the body.

10. The method of claim 9 wherein the honeysuckle contains one or more antiviral compounds, wherein the antiviral compounds benefit the effect of the antibody proteins within the body.
